(19) United States
(12) Patent Application Publication
(10) Pub. No.: US 2002/0192210 A1
(43) Pub. Date: Dec. 19, 2002

(54) Dendritic cell-specific antibodies

(76) Inventors: Derek Nigel John Hart, Dutton Park (AU); Slavica Vuckovic, McKenzie (AU)

Correspondence Address: BAKER & BOTTS, 30 ROCKEFELLER PLAZA, NEW YORK, NY, 10112

(21) Appl. No.: 10/161,298

(22) Filed: Jun. 3, 2002

Related U.S. Application Data
(63) Continuation of application No. PCT/AU00/01486, filed on Dec. 1, 2000.

(30) Foreign Application Priority Data
Dec. 3, 1999 (NZ) 501581

(51) Int. Cl. A61K 39/395; C07K 16/40

(52) U.S. Cl. 424/130.1; 530/388.1

(57) ABSTRACT

The present invention relates generally to immuno-interactive agents which are capable of interacting with an epitope on a molecule in or on dendritic cells (DC). More particularly, the present invention provides immunological reagents such as but not limited to immunoglobulin agents which are capable of interacting with an epitope on a molecule present on or within a subset of DC. Generally, the DC are a subset of DC which have been subjected to differentiation stimulus. The resulting differentiated, activated subset of DC produce a molecule comprising an immunologically detectable epitope which is not detectable in non-differentiated DC. The present invention further relates to cell lines which produce the instant immuno-interactive molecules and to a method for identifying and purifying the above-mentioned subset of DC from a biological sample such as blood using the instant immuno-interactive molecules. The present invention further provides for modulators of the interaction between the instant immuno-interactive molecules and an epitope to which they bind. These modulators are useful in controlling an immune response. The present invention further provides an antigenic molecule or part thereof or cell preparation comprising same which is capable of interacting with the subject immuno-interactive agents. The present invention is further directed to the use of the subject immuno-interactive molecules and/or modulators thereof in the manufacture of medicaments for use in immunomodulation and immunotherapy. An immuno-interactive molecule includes an antibody, fragments thereof and recombinant synthetic or hybrid forms of antibodies.

### FIELD OF THE INVENTION

The present invention relates generally to immuno-interactive agents which are capable of interacting with an epitope on a molecule in or on dendritic cells (DC). More particularly, the present invention provides immunological reagents such as but not limited to immunoglobulin agents which are capable of interacting with an epitope on a molecule present on or within a subset of DC. Generally, the DC are a subset of DC which have been subjected to differentiation stimulus. The resulting differentiated, activated subset of DC produce a molecule comprising an immunologically detectable epitope which is not detectable in non-differentiated DC. The present invention further relates to cell lines which produce the instant immuno-interactive and to a method for identifying and purifying the above-mentioned subset of DC from a biological sample such as blood using the instant immuno-interactive molecules. The present invention further provides for modulators of the interaction between the instant immuno-interactive molecules and an epitope to which they bind. These modulators are useful in controlling an immune response. The present invention further provides an antigenic molecule or part thereof or cell preparation comprising same which is capable of interacting with the subject immuno-interactive agents. The present invention is further directed to the use of the subject immuno-interactive molecules and/or modulators thereof in the manufacture of medicaments for use in immunomodulation and immunotherapy. An immuno-interactive molecule includes an antibody, fragments thereof and recombinant synthetic or hybrid forms of antibodies.

### BACKGROUND OF THE INVENTION

Reference to any prior art in this specification is not, and should not be taken as, an acknowledgment or any form of suggestion that this prior art forms part of the common general knowledge in Australia or any other country.

Bibliographic details of the publications numerically referred to in this specification are collected at the end of the description.

Dendritic cells (DC) constitute a distinct group of potent antigen presenting cells (APC) which are bone marrow derived and found as trace populations in the circulation as well as within both lymphoid and nonlymphoid tissues (1-3). Although their importance as the most effective haemopoietic cell involved in the initiation of primary immune responses has been well demonstrated (4-7), no human DC specific lineage marker has been identified and many features of their ontogeny and relationship to other leukocytes remains unclear.

Plasmacytoid dendritic cells are a distinct subset of human DC found in the bone marrow, blood and in the secondary lymphoid tissues (9). They show a plasma cell-like morphology, lack expression of myeloid markers such as CD13 and CD33, express high amount of IL-3Rα chain (CD123), CD36, chemokine receptor CXCR3, inhibitory receptor ILT3 and L-selectin (CD62L) (9,10). In responses to inflammatory cytokines IP-10 and Mig (11), they migrate through the high endothelial venules (HEV) to secondary lymphoid tissues where they are located around and in close proximity to HEV (10).

A unique feature of these cells is high production of type I interferon (IFN) upon stimulation with bacteria (12), viruses (13,14) or CD40L (10,15) and they are identical to previously described natural IFN-α/β-producing cells (16). IFN-α acts as an autocrine survival factor and maintains about half of the initial number of plasmacytoid DC viable after three days of culture (14). IFN-α produced by DC stimulated by virus promotes production of both IFN-γ and IL10 (14). It also synergizes with IL-12 produced by DC stimulated by CD40L to promote Th1 immune responses and production of IFNγ (15).

By producing large amount of type I IFN plasmacytoid blood CD123⁺DC differentiate subsequently into mature DC. This maturation process involves down-regulation of CXCR3, lost of CD62L expression, increased expression of MHC, co-stimulatory CD80 functional characteristics of DC including potent accessory function and capability to drive Th specific immune responses (14). Differentiation of plasmacytoid DC to mature DC appears to be mediated by TNF-α, which is produced by plasmacytoid DC upon virus stimulation (14). Exogenous added TNF-α also induces (17,18) and anti-TNF-α antibody diminishes differentiation of plasmacytoid DC (17).

Involvement of TNF-α along with high expression of costimulatory and CD83 molecules are events typical for the final stage of maturation of human DC (19). Maturation of plasmacytoid DC, like maturation of other DC, is likely to be a two step process including initial and final stages of maturation (2,20). The initial stage of maturation of DC is dependent on the cytokine GM-CSF, IL-4 and IL3 (19,21,22) and TNF-α and CD40L may provide a further activation signal to complete maturation of DC and increase their T cell stimulatory capacity (15,22).

Despite this phenotypic characterization, identification and, therefore, purification of DC remains difficult as the majority of these antigens are expressed by other resting and activated cell types. Many of the functional and phenotypic features of DC are shared by both Hodgkins cells (HC) and Hodgkins Disease (HD) derived cell lines and there is increasing evidence to support the hypothesis, that in some instances, HC represent a malignant form of 'DC (23-26). Some of these antigens are also shared with activated B lymphocytes (27).

There is a need, therefore, to identify immuno-interactive agents for use in identifying and purifying DC and to discriminate between subsets of DC. There is also a need to identify molecules having epitopes which interact with the above-mentioned immuno-interactive agents. Such molecules are useful for screening for modulators of an immune response. The immuno-interactive agents and modulators thereof are useful in the development of therapeutic applications such as immunotherapy including immunosuppression.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention provides immuno-interactive agents such as in the form of immunological reagents and in particular immunoglobulins which recognize an epitope on a molecule present on or within a subset of DC. The subset of DC represents DC which have been exposed to cytokine-mediated stimulis and are, hence, activated or partially activated.

Accordingly, one aspect of the present invention provides an immuno-interactive molecule comprising an epitope-binding region wherein said epitope is immunodetectably present in stimulated, including partially stimulated, DC and is substantially not immunodetectable in non-stimulated DC.

Another aspect of the present invention is directed to an antibody or epitope-binding fragments thereof wherein said antibody or its fragment is capable of binding to an epitope on a molecule immunodetectably present in stimulated, including partially stimulated DC and is substantially not immunodetectable in non-stimulated DC.

A further aspect of the present invention is directed to a preparation of antibodies or epitope-binding fragments thereof which antibodies or their fragments are capable of binding to an epitope on a molecule immunodetectably present on plasmacytoid DC or myeloid DC stimulated in the presence of GM-CSF and/or IL-3 or other cytokine or functional equivalent but which molecule comprising the epitope is not immunodetectable in non-stimulated plasmacytoid DC or myeloid DC.

Still another aspect of the present invention provides a preparation of antibodies which antibodies are capable of interacting with Ag CMRF-58 immunodetectably present on plasmacytoid DC or myeloid DC subjected to stimulation in the presence of GM-CSF and/or IL-3 or other cytokine or functional equivalent thereof.

Yet another aspect of the present invention is directed to an isolated mAb CMRF-58 or preparation of mAb CMRF-58 antibodies or derivatives thereof which antibody or antibodies exhibit a different immuno-interactive pattern compared to mAb CMRF-44, anti-CD83 antibodies and/or mAb CMRF-56.

Still yet another aspect of the present invention contemplates a method for identifying a subset of DC in a sample, said method comprising contacting said sample with an epitope-binding effective amount of an antibody which antibody is capable of interacting with an epitope present on a molecule in activated DC but is not immunodetectable in non-activated DC which contact being for a time and under conditions sufficient for said antibody to form a complex with said epitope and then detecting the complex.

Even yet another aspect of the present invention contemplates a preparation of DC wherein said preparation substantially comprises CMRF-58⁺ DC and is substantially devoid or depleted of CMRF-58⁻ cells.

Even still another aspect of the present invention provides an immunopotentiating composition comprising a population of DC having a molecule immunodetectably present in activated DC but which molecule is substantially not immunodetectable in non-activated DC, said composition further comprising an antigen capable of generating a protective immunological response to a disease in an animal susceptible to such disease.

Another aspect of the present invention provides an immunomodulating composition comprising agents selected from:

(i) mAb CMRF-58;

(ii) Ag CMRF-58;

(iii) Ag CMRF-58⁺ cells;

(iv) a modulator of Ag CMRF-58-mAb CMRF-58 interaction;

or derivatives or recombinant, synthetic or hybrid forms thereof

which composition further optionally comprises an antigen capable of generating a protective immune response to an infectious agent in an animal such as a mammal including a human.

A further aspect of the present invention further contemplates a method for the treatment or prophylaxis of an animal to a disease condition, said method comprising administering to said animal an effective amount of an immunomodulating composition, said composition comprising agents selected from:

(i) mAb CMRF-58;

(ii) Ag CMRF-58;

(iii) Ag CMRF-58⁺ cells;

(v) a modulator of Ag CMRF-58-mAb CMRF-58 antibodies

or derivatives or recombinant, synthetic or hybrid forms thereof

which composition further optionally comprises an antigen capable of generating a protective immune response to an infectious agent in an animal such as a mammal including a human.

Still another aspect of the invention provides a DC purification system for use in purifying or concentrating DC from a sample containing such cells which includes an antibody or antibody binding fragment as defined above.

Yet another aspect of the present invention consists in differentiated or activated subsets of DC recovered by a process as defined above or by using a purification system as defined above.

Still yet another aspect of the invention provides an immunopotentiating composition comprising activated DC obtained as above and at least one antigen capable of generating a protective immunological response to a disease in an animal such as a mammal including a human susceptible to such disease.

Even yet another aspect of the invention provides an immunopotentiating composition comprising an antibody as defined above.

Even still another aspect of the invention provides an immunopotentiating composition comprising activated DC obtained as above, an antibody as defined above and at least one antigen capable of generating a protective immunological response to a disease in a patient susceptible to such disease.

Another aspect of the invention provides a method of prophylaxis and/or therapy in relation to a disease which comprises administering to a subject susceptible to said disease an immunopotentiating composition as defined above.

A further aspect of the invention provides a method of suppressing an immune response in a patient in need of such treatment comprising the step of administering to said patient an immunosuppressive composition as defined above.

Still another aspect of the invention provides an assay kit which includes mAb CMRF-58 for use as a diagnostic marker of subsets of differentiated or activated DC.

Yet another aspect of the invention provides a method of tolerizing an organ transplant recipient to reduce the risk of rejection of a donated organ upon transplantation, which comprises the step of administering CMRF-58 depleted DC from the organ donor to said recipient.

### BRIEF DESCRIPTION OF THE FIGURES

FIG. 1 is a photographic representation showing plasmacytoid CD123⁺DC differentiate into CD123⁺CMRF58⁺DC with cytokines. Freshly isolated blood Lin⁻ cells, or cultured with cytokines as described in Example 15 were processed for three colors labelling with anti-CMRF58, CD123, and HLA-DR mAb and analyzed by flow cytometry.

FIG. 2 is a graphical representation showing phenotype of CD123⁺CMRF58⁺DC. Blood Lin⁻ cells were cultured with cytokines as described in Example 16. After 12 hours of culture, cells were processed for four color labelling for CMRF58, CD11c, HLA-DR and indicated markers and analyzed by flow cytometry.

FIG. 3 is a graphical representation showing CD123⁺CMRF58⁺DC induce allo-MLR responses. (A) Proliferative response of allo-CD3⁺T lymphocytes (1×10⁵) induced by freshly isolated CD123⁺DC and CD11c⁺DC and (B) cytokine derived CD123⁺CMRF58⁺ and CD11c⁺CMRF58⁺DC. Results are shown as a mean [³H]thymidine uptake count per minute (CPM)±SEM from one of three experiments performed.

FIG. 4 is graphical representation showing CD123⁺CMRF58⁺DC induce KLH specific T cell responses. (A) Stimulation index (SI) of auto-CD4⁺CD45RA⁺ T lymphocytes proliferation induced by KLH (100 μg/ml) pulsed versus non-pulsed freshly isolated CD123⁺DC, CD11c⁺DC, CD14⁺ monocytes and (B) cytokine derived CD123⁺CMRF58⁺ and CD11c⁺CMRF58⁺DC (DC:T cell ratio 1:50). Results are shown as a SI±SEM from one of three experiments performed.

FIG. 5 is a photographic representation showing that CD123⁺CMRF58⁺DC are not present in tonsil. Tonsil Lin⁻ cells were stained with anti-HLA-DR, CD123 and CMRF58 mAb and analysed by flow cytometry. CD123⁺DC are negative (R1) and CD11c⁺DC (R2) are positive for CMRF58.

### DETAILED DESCRIPTION OF THE PREFERRED EMBODIMENTS

The present invention is predicated in part on the identification of an antigenic determinant present in detectable amounts in or on particular DC but is not detectably present in or on certain other populations of DC. The antigenic determinant becomes detectably present on CD123⁺DC plasmacytoid and myeloid CD11c⁺DC following exposure to stimulis such as but not limited to cytokine-mediated stimuli and in particular granulocyte-macrophage colony-stimulating factor (GM-CSF) and/or interleukin-3 (IL-3). The antigenic determinant is considered herein to be an epitope on a molecule on the surface of a DC, embedded in the membrane of a DC or traversing the membrane region and having a cytoplasmic domain and an extracellular domain. Reference herein to plasmacytoid DC preferably includes plasmacytoid CD123⁺DC and reference herein to myeloid DC preferably includes myeloid CD11c⁺DC.

Accordingly, one aspect of the present invention provides an immuno-interactive molecule comprising an epitope-binding region wherein said epitope is immunodetectably present in stimulated, including partially stimulated, DC and is substantially not immundetectable in non-stimulated DC.

Reference herein to an “immuno-interactive” molecule includes any immunological reagent such as but not limited to antibodies including monoclonal antibodies or polyclonal antibodies. Monoclonal antibodies, abbreviated herein as “mAb” are particularly preferred given their homogeneity. Other immunological reagents contemplated herein include antigen-binding fragments of antibodies, recombinant antibodies, hybrid antibodies and synthetic antibodies as well as mixtures of antibodies. Furthermore, the antibodies may be, for example, humanized forms of murine antibodies or otherwise mammalianized forms of other mammalian-derived antibodies. Furthermore, the immuno-interactive molecule may be a T cell-derived antigen-binding molecule (TABM). All these forms of antibodies and antigen-interacting molecules are encompassed by the terms “immuno-interactive agent”, “immunological reagent” and “antibody”. Although the mAb may be any class of antibodies, IgG and IgM antibodies are particularly useful.

The specification also encompasses derivatives of the immuno-interactive molecules and in particular derivatives of antibodies. Derivatives of antibodies include antigen-binding fragments thereof as well as recombinant, synthetic and hybrid forms thereof.

Accordingly, another aspect of the present invention is directed to an antibody or epitope-binding fragments thereof wherein said antibody or its fragment is capable of binding to an epitope on a molecule immunodetectably present in stimulated, including partially stimulated DC and is substantially not immunodetectable in non-stimulated DC.

Reference herein to “immunodetectably present” or “immunodetectable” includes and encompassses the ability to detect an epitope on a molecule within the sensitivity limits of antibody-antigen interactions. The absence of immunodetection is not to necessarily imply that the molecule comprising the epitope is not present but if it is present, its presence at levels below which it can be detected by immunological means.

In one example, immunodetection comprises detecting an antibody labelled with a reporter molecule capable of providing an identifiable signal wherein the antibody has immunointeracted with the epitope on the molecule present in activated DC.

The preferred populations of DC contemplated by the present invention include but are not limited to plasmacytoid and myeloid DC. Furthermore, reference herein to “stimulated” DC includes partial stimulation of DC and in particular stimulation of plasmacytoid or mycloid DC. “Stimulation” in this context is preferably cytokine-mediated stimulation such as but not limited to GM-CSF and/or IL-3-mediated stimulation.

Accordingly, another aspect of the present invention is directed to a preparation of antibodies or epitope-binding fragments thereof which antibodies or their fragments are capable of binding to an epitope on a molecule immunodetectably present on plasmacytoid and/or myeloid DC stimulated in the presence of GM-CSF and/or IL-3 or other cytokine or functional equivalent but which molecule comprising the epitope is not immunodetectable in non-stimulated plasmacytoid or myeloid DC.

Reference to a “preparation” of antibodies includes a population of antibodies subjected to at least one purification, concentration or enrichment step relative to other molecules in a sample. Similarly, an “immuno-interactive molecule”, “immunological reagent” or “antibody” is preferably but not necessarily in isolated form or in a form resulting from at least one purification, concentration or enrichment procedure. Such procedures include centrifugation, adsorption including immunoadsorption, chromatographic separation, precipitation and/or electrophoretic procedures amongst others. The term “preparation” also covers mixtures or blends of antibodies.

In a particularly preferred embodiment, the immunological reagent is monoclonal antibody (mAb) CMRF-58 which recognizes antigen (Ag) CMRF-58 on plasmacytoid or myeloid DC subjected to stimulation in the presence of GM-CSF and/or IL-3 or other cytokine or functional equivalent.

Accordingly, another aspect of the present invention provides a preparation of antibodies which antibodies are capable of interacting with Ag CMRF-58 immunodetectably present on plasmacytoid or mycloid DC subjected to stimulation in the presence of GM-CSF and/or IL-3 or other cytokine or functional equivalent thereof.

Preferably, the plasmacytoid DC and plasmacytoid CD123⁺CMRF-58⁺DC and the myeloid DC are myeloid CD11c⁺CMRF-58⁺DC.

The instant antibody or preparation of antibodies is preferably a monoclonal antibody or preparation of monoclonal antibodies referred to herein as mAb CMRF-58. The antibody may be of any class such as IgG or IgM.

Still another aspect of the present invention is directed to an isolated mAb CMRF-58 or preparation of mAb CMRF-58 antibodies which antibody or antibodies exhibit a different immunointeracting pattern compared to mAb CMRF-44, anti-CD83 antibodies and/or mAb CMRF-56.

Reference herein to an “antibody” includes reference to epitope-binding fragments thereof, hybrid, recombinant and synthetic forms of the antibodies and mixtures of antibodies.

The present invention further provides a hybridoma cell line which produces mAb CMRF-58. This cell line is referred to herein as hybridoma CMRF-58.

The immuno-interactive molecules and in particular mAb CMRF-58 of the present invention have a range of utilities including but not limited to identifying subsets of DC, purifying subsets of DC and screening for modulators which are capable of antagonizing or agonizing interaction between mAb CMRF-58 and Ag CMRF-58. The instant immuno-interactive molecules and the aforementioned modulators may also be able to facilitate a change in the degree of activation of the DC. This is important for immunotherapy including modulating an immune response. Examples of immunomodulation include immunosuppression during transplantation, promoting an immune response or manipulating the activation of DC to render same immunogenic (i.e. non-stimulated) or immunospecific (i.e. after stimulation).

Accordingly, another aspect of the present invention contemplates a method for identifying a subset of DC in a sample, said method comprising contacting said sample with an epitope-binding effective amount of an antibody which antibody is capable of interacting with an epitope present on a molecule in activated DC but is not immunodetectable in non-activated DC which contact being for a time and under conditions sufficient for said antibody to form a complex with said epitope and then detecting the complex.

Generally, but not exclusively, the antibody is labelled with a reporter molecule capable of providing an identifiable signal. Alternatively, the bound antibody is detected using a labelled anti-immunoglobulin antibody. The label may not provide a direct signal but may require the addition of a reagent such as an enzyme or source of light particles.

Preferably, the antibody is mAb CMRF-58 and the epitope is present on Ag CMRF-58 (e.g. an IgG or IgM antibody).

A similar protocol is adopted for purifying and/or concentrating subsets of DC. In this case, the antibody may be first immobilized to a solid support to which a sample comprising DC is contacted. Alternatively, DC comprising an antibody immunointeracted to an antigen are purified by secondary means such as immunological anti-immunoglobulins.

The antibodies of the present invention may be employed in a range of detection systems from the corresponding antigen.

Sandwich assays are among the most useful and commonly used assays and are favoured for use in the present invention. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of Ag CMRF-58 antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of antigen hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention, the sample is one which might contain Ag CMRF-58 including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

In a typical forward sandwich assay, a first antibody having specificity for Ag CMRF-58 or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to about 37° C. including 25° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody.

Alternatively, a second labelled antibody, specific to the first antibody is exposed to the target-first antibody complex to form a target-first antibody-second antibody tertiary complex. The complex is detected by the signal emitted by the reporter molecule.

By “reporter molecule”, as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as but not limited to fluorecein and rhodamine amongst others, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength, the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.

Reference to detecting Ag CMRF-58 includes detecting Ag CMRF-58 alone or following a purification procedure or when present on a DC or DC extract or DC preparation.

The above techniques may be readily applied to the detection of Ag CMRF-58 or CMRF-58⁺ DC.

The present invention further contemplates a preparation of DC wherein said preparation substantially comprises CMRF-58⁺ DC and is substantially devoid or depleted of CMRF-58⁻ cells.

The antibodies of the present invention are particularly useful in providing an immunopotentiating composition.

Accordingly, another aspect of the present invention provides an immunopotentiating composition comprising a population of DC having a molecule immunodetectably present in activated DC but which molecule is substantially not immunodetectable in non-activated DC, said composition further comprising an antigen capable of generating a protective immunological response to a disease in an animal susceptible to such disease in an animal such as a mammal including a human.

Preferably, the population of DC comprises Ag CMRF-58⁺ cells immuno-interactive with mAb CMRF-58.

The composition may also comprise an antagonist or agonist of Ag CMRF-58-mAb CMRF-58 interaction.

Preferably, the disease is an infection by a microbial, fungal, yeast or lower cellular animal.

The term “animal” includes a mammal such as a human, primate, livestock animal (e.g. sheep, cows horses, pigs), laboratory test animals (e.g. mice, rats, rabbits, guinea pigs), companion animals (e.g. cats, dogs) or captive wild animals.

Most preferably, the animal is a human.

In a particularly preferred embodiment, there is provided an immunomodulating composition comprising agents selected from:

(i) mAb CMRF-58;

(ii) Ag CMRF-58;

(iii) Ag CMRF-58⁺ cells;

(iv) a modulator of Ag CMRF-58-mAb CMRF-58 interaction;

or derivatives or recombinant, synthetic or hybrid forms thereof

which composition further optionally comprises an antigen capable of generating a protective immune response to an infectious agent in an animal such as a mammal including a human.

An immunomodulating composition may have immunoactivation properties or immunosuppression properties.

The present invention further contemplates a method for the treatment or prophylaxis of an animal to a disease condition, said method comprising administering to said animal an effective amount of an immunomodulating composition, said composition comprising agents selected from:

(i) mAb CMRF-58;

(ii) Ag CMRF-58;

(iii) Ag CMRF-58⁺ cells;

(iv) a modulator of Ag CMRF-58-mAb CMRF-58 interaction

or derivatives or recombinant, synthetic or hybrid forms thereof

which composition further optionally comprises an antigen capable of generating a protective immune response to an infectious agent in an animal such as a mammal including a human human.

The immunomodulating composition may also comprise an antagonist or agonist of mAb CMRF-58, Ag mAb CMRF-58 interaction. Such an antagonist or agonist may be readily identified using natural product screening, or screening by chemical libraries or modifying Ag CMRF-58 or mAb CMRF-58.

Accordingly, the present invention contemplates a composition comprising an immuno-interactive molecule specific for Ag CMRF-58.

The composition forms suitable for injectable use include sterile aqueous solutions (where water soluble) and sterile powders for the extemporaneous preparation of sterile injectable solutions. It must be stable under the conditions of manufacture and storage and must be preserved against the contaminating action of microorganisms such as bacteria and fungi. The carrier can be a solvent or diluent containing, for example, water, ethanol, polyol (for example, glycerol, propylene glycol and liquid polyethylene glycol, and the like), suitable mixtures thereof and vegetable oils. The preventions of the action of microorganisms can be brought about by various anti-bacterial and anti-fungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the immuno-interactive molecules in the required amount in the appropriate solvent or diluent as followed by sterilization such as by filter sterilization. In the case of sterile powders for the preparation of sterile injectable solutions, the preferred methods of preparation include vacuum drying and the freeze-drying technique which yield a powder of the immuno-interactive molecule plus any additional desired ingredient from previously sterile-filtered solution thereof.

Pharmaceutically acceptable carriers and/or diluents include any and all solvents, dispersion media, coatings, anti-bacterial and anti-fungal agents, isotonic and absorption delaying agents and the like. The use of such media and agents for pharmaceutical active substances is well known in the art and except insofar as any conventional media or agent is incompatible with the immunointeractaive molecule, their use in the therapeutic compositions is contemplated. Supplementary active ingredients can also be incorporated into the compositions.

The principal immuno-interactive molecule will be added in a concentration effective to interact to a Ag CMRF-58 and inhibit or reduce the function of the Ag CMRF-58. For example, an effective amount may range from about 10 ng to about 2000 mg, or 50 ng to about 1000 mg or 100 ng to about 500 mg or about 1 ng to about 50 mg such as but not limited to about 5 mg. Generally, but not exclusively, the dosage is given over a set time such as per day.

In still a further aspect, the invention provides a DC purification system for use in purifying or concentrating DC from a sample containing such cells which includes an antibody or antibody binding fragment as defined above.

Conveniently, the purification system is designed to purify subsets of differentiated or activated human DC and the antibody is optionally labelled mab CMRF-58.

In still a further aspect, the present invention consists in differentiated or activated subsets of DC recovered by a process as defined above or by using a purification system as defined above.

Preferably, said subsets are a CMRF-58⁺ subset and a CMRF-58⁻ (or CMRF-58⁺ depleted) subset.

In yet a further aspect, the invention provides an immunopotentiating composition comprising activated DC obtained as above and at least one antigen capable of generating a protective immunological response to a disease in an animal susceptible to such disease.

In still a further aspect, the invention provides an immunoactive composition comprising an antibody as defined above. Said immunoactive composition can be an immunopotentiating composition or an immunosuppressive composition.

The immunopotentiating composition may comprise an antibody as defined above and at least one antigen capable of generating a protective immunological response to a disease in a patient susceptible to such disease.

In still a further aspect, the invention provides an immunopotentiating composition comprising activated DC obtained as above, an antibody as defined above and at least one antigen capable of generating a protective immunological response to a disease in a patient susceptible to such disease.

In still a further embodiment, the invention provides a method of prophylaxis and/or therapy in relation to a disease which comprises administering to a subject susceptible to said disease an immunopotentiating composition as defined above.

In still a further embodiment, the invention provides a method of suppressing an immune response in a patient in need of such treatment comprising the step of administering to said patient an immunosuppressive composition as defined above.

In yet a further aspect, the invention provides an assay kit which includes mAb CMRF-58 for use as a diagnostic marker of subsets of differentiated or activated DC.

In yet a further embodiment, the invention provides a method of manipulating an immune response to facilitate organ transplantation to reduce the risk of rejection of a donated organ upon transplantation, or to manipulate the immune response to reduce graft failure and graft versus host disease as a consequence of allogeneic bone marrow transplantation which method comprises the step of administering CMRF-58 depleted DC from the organ donor to said recipient.

In a further aspect, the instant invention provides Ag CMRF-58 in isolated form or in a preparation of DC, said antigen having an ability to bind mAb CMRF-58.

In one embodiment, the antigen also has a different binding pattern to mAb CMRF-44, mAb CMRF-56 and/or anti-CD83 antibodies.

In another embodiment, one form of an antibody to Ag CMRF-58 does not bind Ag CMRF-44, Ag CMRF-56 and/or CD83.

It will be appreciated that the antibodies which bind the Ag CMRF-58 can be in the form of antisera containing polyclonal antibodies or, as is preferred, monoclonal antibodies may be obtained by use of hybridoma technology. Still further, antibodies or binding fragments can be produced using biochemical or recombinant DNA techniques.

It is most desirable for the immunological reagents of the invention to be monoclonal antibodies or binding fragments of such antibodies. The general procedure of Kohler and Milstein (28) is therefore used. Generally, this procedure involves obtaining antibody-producing cells from the animal and fusing the antibody-producing cells with strains of myeloma cells to produce hybridomas. These hybridomas are grown or cultured to produce monoclonal antibodies specific for DC.

An example of the procedure using myeloma cell line NS-1 is given below. Cell line NS-1 is obtainable from Professor C Milstein, MRC Laboratory of Molecular Biology, Hills Road, Cambridge CB2 2QH, United Kingdom.

Other myeloma cell lines are known in the art and include, for example, the following cell lines: X63Ag8 653, SP2/0, FO and NSO/1. Cell lines which neither synthesize nor secrete immunoglobulin heavy or light chains (e.g. SP2/0) are generally preferred to cell lines which synthesize but do not secrete, immunoglobulin chains.

If desired, antibody fragments can be prepared by controlled protease digestion of whole immunoglobulin molecules as described by Tjissen (29).

Alternatively, antibody fragments can be prepared using molecular biological techniques by isolating, from hybridoma cells, the genetic material encoding the variable regions of the heavy, light or both chains of the monoclonal antibodies and expressing them in suitable organisms for the product of recombinant antigen binding fragments (Fv, ScFv, Fab etc.) of the monoclonal antibody (30).

By way of illustration of the invention, the generation and characterization of a monoclonal antibody, designated mAb CMRF-58, capable of binding to an epitope on a differentiation/activation Ag CMRF-58 of human dendritic cells will now be described. From this description, those persons skilled in this art will also appreciate how other antibodies (or their binding fragments) which bind to Ag CMRF-58 can be obtained for use in the extraction of human DC or DC from other animals.

There are a number of uses to which the antibodies of the invention (which recognize and bind to the activation Ag CMRF-58) can be put. Such uses include (1) the identification (for diagnostic purposes) of subsets of DC, and (2) the purification/concentration of subsets of DC, and these uses accordingly represent further aspects of this invention.

Diagnostic applications of the present exemplary mAb CMRF-58 include allowing for assessment of certain DC subsets (CMRF-58 positive) against other (CMRF-58 negative) DC subsets, which may be of use in the diagnosis and/or therapy of diseases such as cancer.

In such applications, any immunological-based assay procedures known in the art can be employed for quantifying the amount of activated DC in a sample. Such procedures are summarised in Tijssen (29) such as flow cytometry, ELISA, RIA and fluorescence microscopy among others.

In terms of isolation of activated DC, once again any process or purification system which employs the antibodies (or their binding fragments) as the primary immunological reagent can be used. Many such processes are known, as are purification systems which allow for these processes to be put into effect. An example of such a purification system is the avidin-biotin immunoaffinity system (31) (U.S. Pat. Nos. 5,215,927, 5,225,353, 5,262,334, 5,240,856 and PCT/US91/07646 published Apr. 30, 1992, all incorporated herein by reference). This system employs directly or indirectly a biotinylated monoclonal antibody directed against a target cell and a column containing immunobilized avidin and can be readily adapted to extract activated human DC, in this case from human peripheral blood, using the exemplary mAb CMRF-58 as follows:

(1) A sample of human peripheral blood containing the human DC is mixed with biotinylated mAb CMRF-58 and incubated to allow formation of mAb CMRF-58/human DC complexes.

(2) Following incubation, the mixture is introduced into a continuous-flow immunoadsorption column filled with avidin-coated beads, the strong affinity between biotin and avidin causing the biotin-coated mAb CMRF-58 (together with the human DC to which they have bound) to adhere to the avidin-coated beads.

(3) After unwanted cells present in the mixture are washed away, captured differentiated activated human DC are removed from the column by gentle agitation and are available for use.

(4) As an alternative CMRF-58 depleted DC preparations might be used to obtain an alternative DC subset for therapeutic purposes.

Variations on this theme using mAb CMRF-58 as primary antibody (to bind to activated DC) and a biotinylated secondary antibody (to bind to mAb CMRF-58) can also be employed.

It will be appreciated that before admixture with mAb CMRF-58 in accordance with the above protocol, the human peripheral blood sample should be treated to ensure that the DC the sample contains are activated. This can easily be achieved by, for example, overnight incubation of the sample (preferably in the presence of an active agent such as GM-CSF).

For use in the above protocol, mAb CMRF-58 can be biotinylated by any one of a number of conventional methods. For example, the biotinylation procedure of Berenson et al. (31) can be employed.

A possible and preferred preliminary step in the methods outlined above is the enrichment of DC in the sample by gradient centrifugation (32-34). While this optional enrichment step can employ any suitable known gradient medium (such as albumin or metrizamide), it is, however, preferred that a Nycodenz medium (Nycomed Pharma, Oslo, Norway) be used (35) in relation to 16 hour cultured T lymphocyte-depleted peripheral blood mononuclear cells. The applicants have found that use of this gradient reliably yields a population of low density cells that is highly enriched for DC.

Variation on this theme using depletion of T,B,monocytes and NK cells to enriched DC can also be employed.

It will be apparent to one skilled in the art that there are numerous other means of immunoselection of dendritic cells, in addition to avidin-biotin immunoaffinity chromatography. These include, but are not limited to, immunoselection using magnetic beads, cell sorting, ferrofluids, dipsticks, petri dishes, and a wide variety of other solid phases that can be derivatized so as to specifically bind mAb CMRF-58 labelled DC.

Once purified/concentrated by the above or any other suitable process, the differentiated/activated DC can be employed in research or in commercial applications. One such potentially commercial application for activated DC is as part of an immunopotentiating composition together with an antigen protective against disease, for either prophylaxis or therapy. It is believed that such compositions would increase both the speed and efficiency of the immune response generated against the protective antigen.

Equally, CMRF-58 depleted or negative DC have application in tolerizing a recipient of a donated organ to reduce the risk of rejection. Tolerization can be achieved by obtaining the CMRF-58 negative subset of DC from the donor of the organ and administering those DC to the recipient. Generally, this will occur pre-transplant.

Other applications of the activated DC (either CMRF-58 positive or negative) will of course be apparent to those persons skilled in this art.

Another application of the mAb CMRF-58 is as a component of a composition to induce immunosuppression. Such a composition can comprise the monoclonal antibodies alone, but for efficiency will generally include an effector molecule coupled to the antibody which induces DC death. The effector molecule may be a toxin (such as diphtheria toxin or ricin A chain) or an apoptotic signalling molecule.

Deposit

Hybridoma CMRF-58 (produced using myeloma cell line NS-1) has been deposited to provide supplemental disclosure of the invention. Deposition was with Deutsche Sammlung Von Mikroorganismen und Zellkuturen GmbH (DSMZ), Mascheroder Weg lb, D-38124 Braunschweig, Germany, with the date of deposition being Dec. 2, 1999 and under Accession Number DSM ACC2434.

The present invention is further described by the following non-limiting Examples.

### EXAMPLE 1

### Tissue

Units of blood (400 ml) were obtained from normal healthy volunteers, who were well at the time of blood collection. Tonsils were obtained by routine tonsillectomies.

### EXAMPLE 2

### Generation of Monoclonal Antibodies (mAb) CMRF-58

Newborn balb/c mice (<24 hours) were tolerized to common cellular antigens by intraperitoneal injection with the monocytoid cell line U937. Three and half month old tolerized mice were immunized intraperitoneally/subcutaneously with the B cell line Raji and splenocytes fused with the myeloma line NS-1, five days later. Following cell fusion, clones producing mAb reactive with the cell line Raji were identified. Hybridoma AIF2 was isolated. Antibody secreted by this hybridoma designated as CMRF-58 reacts with approximately 30% fresh B and almost all cultured B cells (RPMI/10% v/v fetal calf serum (FCS),12 hours), but not with T, NK cells and monocytes. The mAb CMRF-58 reacts with a small population of fresh isolated blood Lin⁻DR⁺DC (5-8%), and with the majority of Lin⁻DR⁺DC (50-70%) upon culture (GM-CSF/IL-3, 12 hours). Also, CMRF-58 mAb shows reactivity with a small population (10-20%) of monocyte derived DC (Mo-DC) and with all Mo-DC maturated with LPS. All together, the data suggest that CMRF-58 mAb is specific for antigen expressed during maturation of B cells and DC.

### EXAMPLE 3

### Enzyme Studies

The enzyme susceptibility of the antigen recognized by CMRF-58 mAb was tested by incubating the cell lines Raji and KM-H2 (0.5×10⁶ ml) in 1 ml PBS containing either pronase (25 μg/ml, 40 min, 37° C.), trypsin (5, 10 μg/ml, 15 min, 37° C.) or neuraminidase (0.1 U/ml, 15 min, 37° C.). Reaction was stopped and enzyme was moved by washing cells three times with cold PBS/10% v/v FCS. Cells incubated with enzyme or with PBS only were labelled with CMRF-58 or control mAb and analyzed by flow cytometry.

### EXAMPLE 4

### Monoclonal Antibodies and Flow Cytometry

Antibody CMRF-58 (IgM) was produced in the inventors' laboratory. Antibody against: CD3 (OKT3, IgG₂ₐ), CD8 (OKT8, IgG₂ₐ), CD45RO (UCHL-1, IgG₂ₐ), HLA-DR (L243, IgG₂ₐ), CD11b (OKM1, IgG₁) obtained from ATCC (Rockville, Md.). Antibody against: CD19 (FMC63, IgG₁) was obtained from Professor H. Zola (Adelaide, Australia), CD14 (CMRF31, IgG₂ₐ) was obtained produced in the inventors' laboratory and CD16 (HuNK2, IgG₂ₐ) was from Professor I. McKenzie (Melbourne, Australia). Phycoerythrin (PE)-conjugated mAb against CD7 (M-T701, IgG₁), CD4 (SK3, IgG₁), CD11c (S-HCL-3, IgG_{2b}), CD34 (My10, IgG₁), CD33 (P67.6, IgG₁) negative controls mAb IgG₁, IgG_{2b}, allophycocyanin (APC)-conjugated mAb against CD11c (S-HCL-3, IgG_{2b}), FITC-conjugated avidin (AV-FITC) all were purchased from BDIS (Sydney, Australia). PE-conjugated mAb against CD40 (MAB89, IgG₁), CD20 (HRC20, Ig2a), CD56 (NKH-1, IgG₁), CD83 (HB15, IgG_{2b}) all were purchased from Coulter-Immunotech (Sydney, Australia). PE-conjugated mAb against CD86 (IT2.2, IgG_{2b}), FITC-conjugated and PE-conjugated mAb against CD123 (7G3, IgG₂ₐ), negative control mAb IgG_{2b} were purchased from PharMingen (Sydney, Australia). Phycoerythrin-cyanine 5.1 (PC5)-conjugated mAb against HLA-DR (Immu-357, IgG₁), and negative controls mAb PC5-IgG₁ were purchased from Coulter Immunotech (Coulter Electronics, Sydney, Australia). FITC-conjugated sheep antimouse immunoglobulin (FITC-SAM) was purchased from AMRAD (AMRAD, Victoria, Australia). Three or four color immunofluorescence staining and analyzing was performed. Cell analyzed using a FACSCalibur and FACSVantage (BDIS, Sydney, Australia).

### EXAMPLE 5

### Media, Cytokine and Reagents

Except where specifically noted, cells where cultured in medium RPMI 1640 supplemented with 100 U/ml penicillin, 100 μg/ml streptomycin, 2 mM L-glutamine and 10% v/v FCS(Life Technologies, Melbourne, Australia). Recombinant human cytokines GM-CSF was purchased from Sandoz-Pharma (Sidney, Australia) and IL-3 from Gibco (Life Technologies, Melbourne, Australia). Keyhole limpet hemocyanin (KLH), biotin conjugated lectin peanut agglutinin (PNA) and mouse serum were purchased from Sigma (St Louis, Mo.). Neuraminidase was purchased from Behring (Marburg, Germany) and pronase from Boehringer Mannheim (Mannheim, Germany).

### EXAMPLE 6

### Cell Preparation

Total blood PBMC were depleted of leukocytes other than DC and basophils by a mixture of mAb against CD3, CD19, CD14, CD11b and CD16, magnetic beads (PerSeptive Biosystems, Mass.) and sorting separation (FACSVantage, BDIS, Sydney, Australia). Sorted Lin⁻ cells almost exclusively contained HLA-DR⁺ (>90%), witch includes approximately 10% CD34⁺ cells, less than 10% contamination by Lin⁺ cells (CD7⁺,CD20⁺,CD64⁺,CD56⁺) and approximately 30% CD11c⁺DC and 50% plasmacytoid CD123⁺DC. From the same Lin⁻ cell preparation, plasmacytoid DC were sorted as CD4⁺CD11c⁻Lin⁻ and CD11c⁺DC were sorted as CD11c⁺CD4⁻Lin⁻. Tonsil samples were processed immediately and a single cell suspension prepared by mincing the tissue finely and passing the material through a wide mesh sieve. Lin⁻ cells were isolated as described above for blood Lin⁻ cells. Allogeneic CD3⁺T lymphocytes were purified by a mixture of mAb against CD19, CD14, CD11b, CD16, HLA-DR and magnetic bead separation. Naïve CD4⁺CD45RA⁺ lymphocytes were purified by including mAb against CD8 and CD45RO in the mAb mixture used for isolation of allogeneic CD3⁺ T lymphocytes and magnetic bead separation.

### EXAMPLE 7

### Cell Culture

Sorted Lin⁻ cells were cultured at a density of 0.5×10⁶/ml in polypropylene tubes (Falcon, Becton Dickinson, Sydney, Australia) for 12 hours in RPMI/10% v/v FCS supplemented with GM-CSF 200 U/ml, IL-3 10 ng/ml. Number of viable cells (>90%) were assessed by trypan blue. From the same culture plasmacytoid DC were sorted as CMRF-58⁺CD11c³¹ and CD11c⁺DC were sorted as CD11c⁺CMRF-58⁺ cells.

### EXAMPLE 8

### Transmission Electron Microscopic

Sorted fresh or cytokine(s)-derived plasmacytoid (CD123⁺CMRF-58⁻) DC and CD11c⁺(CD11c⁺CMRF-58⁻, CD11c⁺CMRF-58⁺) DC were fixed in 3% v/v glutaraldehyde plus 4% v/v paraformaldehyde 0.8% w/v calcium chloride in 0.1% M sodium cacodylate buffer pH 7.4, post fixed in 1% aqueous OsO4, stained en block with 5% aqueous uranyl acetate, dehydrated in graded ethanol solution and embedded in Epon/Araldite epoxy resin. Ultrathin sections were cut using a Leica UCT ultramicrotome stained in lead citrate and observed in a JEOL 1200EXII TEM.

### EXAMPLE 9

### Functional Analysis MLR

Graded number of freshly isolated or cytokine derived plasmacytoid DC and CD11c⁺DC were co-cultured with 1×10⁵ allogeneic CD3⁺T lymphocytes in RPMI/10% v/v FCS in 96-well round-bottom microtiter plates (Falcon Becton Dickinson, Sydney, Australia), for 5 days. During the last 18 hours, cells were pulsed with 0.5 μCi [³H]thymidine (Amershem Pharmacia Biotech, Sydney, Australia) and incorporation of the radionucleotide was measured by β-scintillation counter (Wallac, Sydney, Australia). Background of controls (CD3⁺T lymphocytes or DC alone) were always <200 cpm.

### EXAMPLE 10

### KLH Specific T Cell Proliferation

Freshly isolated of cytokine derived plasmacytoid DC or CD11c⁺DC were incubated for 3 hours with KLH 100 μg/ml washed twice and cocultured (2000 DC/well) with 1×10⁵ CD4⁺CD45RA⁺ autologous T lymphocytes in RPMI/10% v/v FCS in 96-well round-bottom microtiter plates, for 7 days. During the last 18 hours of culture, cells were pulsed with 0.5 μCi [3H]thymidine and processed as described for MLR experiments. Background of controls (CD4⁺ T lymphocytes alone, DC alone, CD4⁺ T lymphocytes plus Ag, DC plus Ag) were always <200 cpm.

### EXAMPLE 11

### IFN-α Induction

The Staphylococcus aureus [SAC], purchased from ATTC (ATTC number 25923) was used as IFN-α inducer. SAC grown and heat treated according to published protocol (12). Approximately 5×10⁶ heat killed SAC co-cultured with 5000-10000 sorted fresh isolated or cytokine derived plasmacytoid DC and CD11c⁺DC in 0.2 ml total volume in RPMI supplemented with 2 mM L-glutamine and 10% v/v FCS without penicillin and streptomycin in 96-well flat bottom microtiter plates, in duplicate or triplicate cultures. In control cultures, DC were co-cultured without SAC. After 20-24 hours of culturing at 37° C. and 5% CO₂ in air, cells collected and analyzed for IFN-α mRNA.

### EXAMPLE 12

### Reverse Transcriptase (RT)-PCR

RNA was isolated from cells with TRIzol (Life Technologies, Rockville, Md., USA) as per the manufacturer's instructions. DNA contamination was removed by digestion with deoxyribonuclease I (Roche Molecular Biochemicals, NSW, Australia). Reverse transcription was carried out with 18 mer oligo dT adaptor primer with Superscript II RT (Life Technologies, Rockville, Md., USA). 20 μl PCR reactions were performed to detect IFN-α as described (13). Control PCR reactions were performed in the same manner with GAPDH primers.

### EXAMPLE 13

### Immunohistology

Immunofluorescence double labelling of acetone fixed tonsils tissue sections was carried out as described above for cell suspension. Observation was made with Olympus BX60 immunofluorescence microscope and images were captured using Power Mac equipped with Macprobe software.

### EXAMPLE 14

### Enzsyme Susceptibility of the Antigen Recognized by CMRF58 mAb

Enzyme studies performed on the cell lines Raji and KM-H2 showed that binding of CMRF-58 mAb to its antigen was not affected with pronase or trypsin treatment which caused the complete loss or decrease of anti-CD4 mAb binding to its antigen on KM-H2. Binding of CMRF-58 mAb was also not affected with neuraminidase treatment which caused the increased binding of biotin conjugated lectin PNA to its ligand on the Raji and KM-H2. These results indicate that CMRF-58 antigen is resistant to proteases and neuraminidase, suggesting that the antigen is not a glycoprotein.

### EXAMPLE 15

### Blood Plasmacytoid DC Mature into CD123⁺CMRF-58⁺DC in Presence of Cytokines

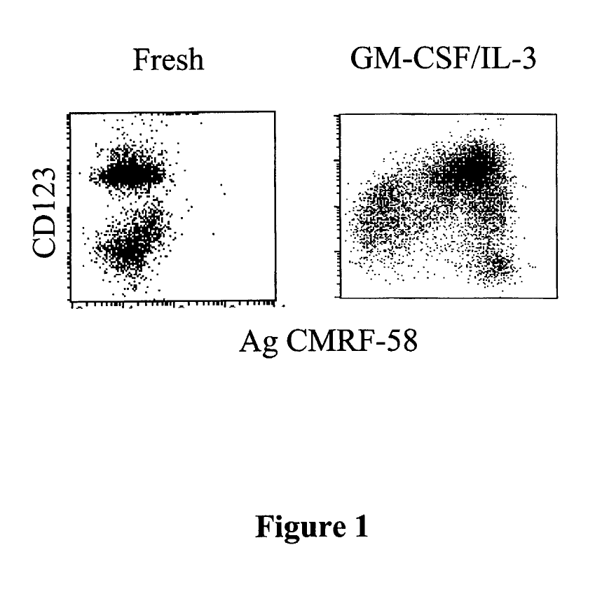

Maturation of blood plasmacytoid DC was monitored after 12 hours culture of Lin⁻ blood cells which were prepared using a combination of immunomagnetic depletion of lineage positive cells and cell sorting (see Example 6). When cultured with GM-CSF or with IL-3 alone or with both of them, Lin⁻ cells survived with viability >90% indicating, as previously shown (18) that GM-CSF alone is sufficient for survival of blood DC. Phenotypic analysis of freshly isolated blood DC showed lack of CMRF-58 antigen expression on plasmacytoid CD123⁺DC (FIG. 1). Upon culture with GM-CSF and IL-3, CMRF-58 antigen was induced on plasmacytoid CD123⁺DC. GM-CSF or IL-3 alone could also induce expression of CMRF-58 antigen on plasmacytoid CD123⁺DC. Expression of this antigen was not restricted to CD123⁺DC it was also induced on CD11c⁺DC (CD123^{dim/neg} cell in FIG. 1). These data demonstrated that the cytokine GM-CSF or IL-3 can induce rapid maturation of plasmacytoid blood DC from resting CD123⁺CMRF-58⁻ into CD123⁺CMRF-58⁺ phenotype.

### EXAMPLE 16

### Cytokine Derived CD123⁺CMRF-58⁺ DC have Immature Phenotype

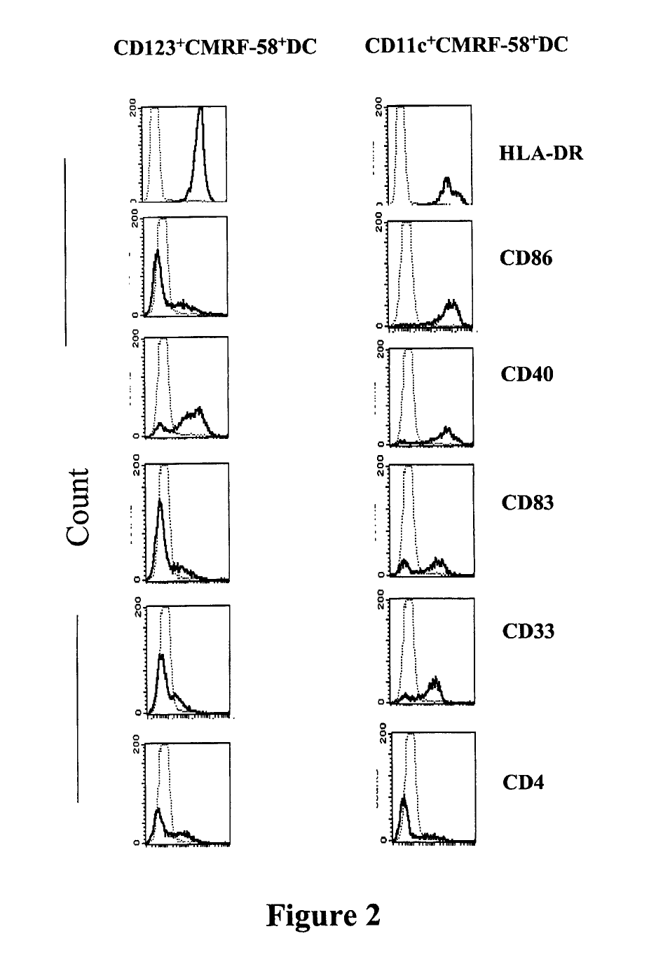

Phenotypic analysis revealed that cytokine derived CD123⁺CMRF-58⁺DC are HLA-DR^{hi}, express CD40 but do not develop expression of co-stimulatory CD86 or activation CD83 molecules (FIG. 2). In addition, CD123⁺CMRF-58⁺ DC showed lack of expression of myeloid CD33 and down-regulated expression of CD4 molecules. In contrast, CD11c⁺ DC from the same culture, developed a phenotype typical of mature DC with CD86 and CD83 expression, retained their expression of CD33 and did not develop expression of CD4 molecules. Both, freshly isolated plasmacytoid CD123⁺DC and cytokine derived CD123⁺CMRF-58⁺DC are 5-6μ in diameter, possess polimorphic nuclei with marginal heterochromatin and a prominent nucleolus. Parallel arrays of rough endoplasmic reticulum (RER) marginated towards the plasma membrane were observed in both of them and no obvious changes in cell morphology were noted between them. For comparison, numerous short dendritic processes occur on the plasma membrane of fresh isolated CD11c⁺DC, and these processes are elongated on cytokine derived CD11c⁺CMRF-58⁺ DC.

### EXAMPLE 17

### Cytokines Derived CD123⁺CMRF-58⁺DC Acquired Accessory Function and Capability to Prime Naïve Antigen Specific T Cell Responses

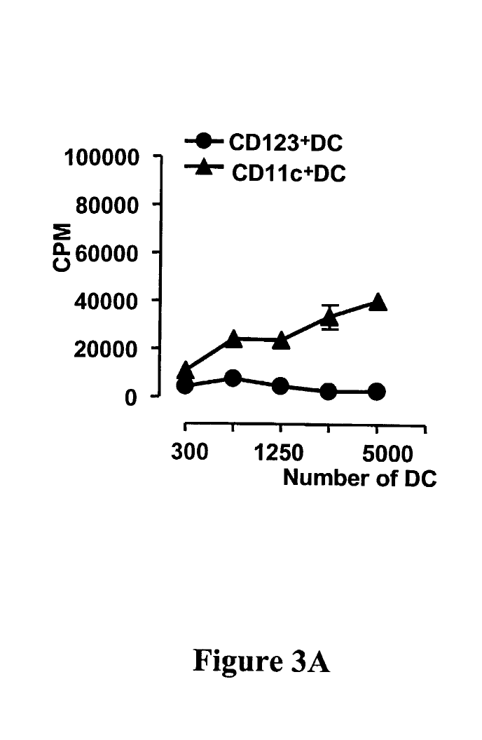

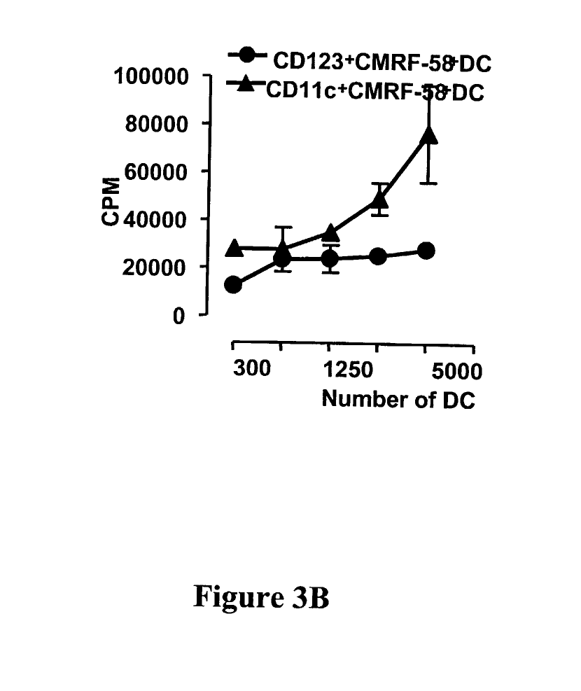

To test the functional properties of cytokine derived CD123⁺CMRF-58⁺ DC, their accessory cell potential was analyzed. Consistent with previous report (17,23) freshly isolated CD123⁺DC were incapable of inducing proliferative MLR responses, in contrast to CD11c⁺DC (FIG. 3A). Cytokine derived CD123⁺CMRF-58⁺DC stimulate MLR responses but to a lesser extent compared to the CD11c⁺DC derived from the same culture (FIG. 3B).

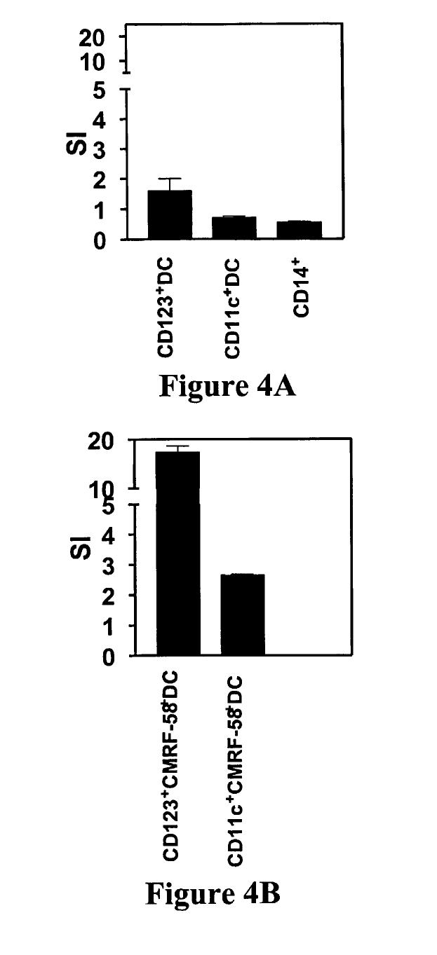

Freshly isolated CD123⁺DC, CD11c⁺DC or cytokine derived CD123⁺CMRF-58⁺ and CD11c⁺CMRF-58⁺DC were pulsed with KLH and incubated with naïve autologous CD4⁺CD45RA⁺ T cells. Freshly isolated CD123⁺DC similar to CD11c⁺DC or CD14⁺ monocytes induced less pronounced or, even, no KLH-dependent T cell responses (FIG. 4A). In contrast, cytokine derived CD123⁺CMRF58⁺DC were capable to induce remarkable KLH-dependent T cell proliferation (FIG. 4B). It appears that CD123⁺CMRF-58⁺DC differ significantly to CD11c⁺CMRF-58⁺ DC derived from the same culture in their ability to stimulate naive antigen specific T cell proliferation. These results confirmed that plasmacytoid CD123⁺DC in the CD123⁺CMRF-58⁺ stage of differentiation acquire features typical of professional antigen presenting cells, including accessory functions and the capability to induce antigen specific naïve T cell responses.

### EXAMPLE 18

### Cytokines Derived CD123⁺ CMRF-58⁺ Cells are Poor IFN-α Producers

Freshly isolated CD123⁺DC, CD11c⁺DC or cytokine derived CD123⁺CMRF-58⁺ and CD11c⁺CMRF-58⁺DC were stimulated with heat killed SAC for IFN-α production. Analysis of IFN-α production by PCR showed that SAC induced high levels of IFN-α mRNA in freshly isolated plasmacytoid CD123⁺DC but not in CD11c⁺DC. Low levels of IFN-α mRNA was detected in culture of plasmacytoid CD123⁺DC without SAC. Cytokine derived CD123⁺CMRF-58⁺DC produced low or no IFN-α mRNA in response to heat killed SAC. No IFN-α mRNA was detected in cytokine induced CD11c⁺CMRF-58⁺DC. Thus cytokine derived CMRF-58⁺CD123⁺DC retained low or are loosing their capability to produce IFN-α in response to bacteria.

### EXAMPLE 19

### CD123⁺CMRF-58⁺DC are not Detected in Tonsils

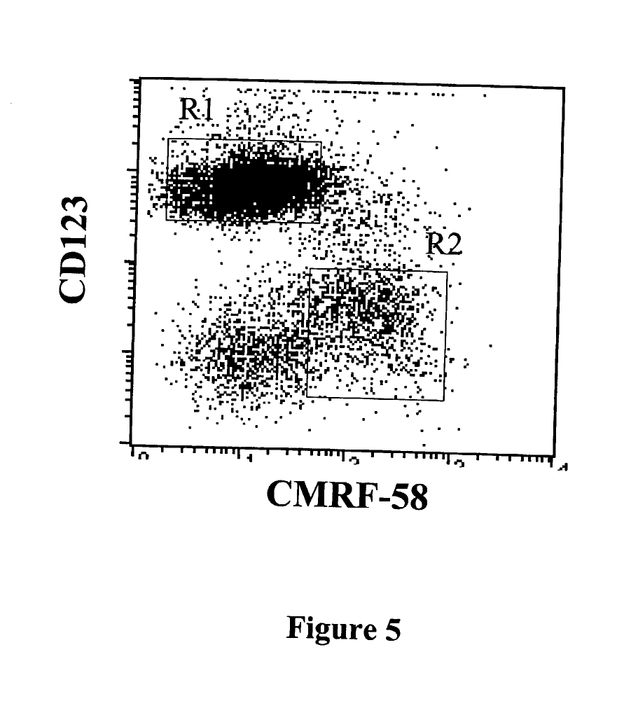

To test whether CD123⁺DC acquired a differentiation phenotype in vivo similar to that induced by cytokines in vitro, the inventors stained tonsil cell suspension and tonsil section. As shown in FIG. 5 CD123⁺DC in a fresh preparation of tonsil mononuclear cells do not express CMRF58 antigen. Accordingly, immunostaining of tonsil section most of CD123⁺DC are located around HEVs and they do not express CMRF-58 antigen (color photographs of tissue staining are available on request). It appears that CD123⁺DC migrate to lymohoid tissue as immature CD123⁺CMRF58⁻DC. Another possibility is that expression of CMRF-58 is a transient event during differentiation of plasmacytoid CD123⁺DC. In contrast to plasmacytoid DC, tonsil CD11c⁺DC express CMRF-58 antigen and the CD11c⁺CMRF-58⁺DC were found in tonsil cell suspensions and tonsil sections (color photographs of tissue staining are available on request).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

Bibliography

1. Steinman, R. M., 1991, The dendritic cell system and its role in immunogenicity. Ann. Rev. Immunol. 9: 271.

2. Hart, D. N. J., 1997, Dendritic cells: Unique leucocyte populations which control the primary immune response. Blood 90: 3245-3287.

3. Macpherson, G. G., 1989, Lymphoid dendritic cells: their life history and roles in immune responses. Res. Immunol. 140: 877.

4. Steinman, R. M., Gutchinov, B., Witmer, M. D. and Nussenweig, M. C., 1983, Dendritic cells are the primary stimulators of the primary mixed lymphocyte reaction in mice. J. Exp. Med. 147: 613.

5. Inaba, K., Witmer-Pack, M. D. and Steinman, R. M., 1984, Clustering of dendritic cells, helper T lymphocytes and histocompatibility B cells during primary T cell responses in vitro, J. Exp. Med. 160:858.

6. Kuntz Crow, M. and Kunkel, H. G., 1982, Human dendritic cells: major stimulators of the autologous and allogeneic mixed leucocyte reactions. Clin. Exp. Immunol. 49: 338.

7. Thomas, R., Davis, L. S. and Lipsky, P. E., 1993, Isolation and characterization of human peripheral blood dendritic cells. J. Immunol. 150: 821.

8. Hart, D. N. J. and McKenzie, J. L., 1988, Isolation and characterization of human tonsil dendritic cells. J. Exp. Med. 168: 157.

9. Olweus, J., BitMansour, A., Warnke, R., Thompson, P. A., Carballido, L. J. and Lund-Johansen, F., 1997, Dendritic cell ontogeny: a human dendritic cell lineage of myeloid origin. Proc. Natl. Acad. Sci USA 94: 12551.

10. Cella, M., Jarrosassay, D., Facchetti, F., Alebardi, O., Nakajima, H., Lanzavecchia, A. and Colonna, M., 1999, Plasmacytoid monocytes migrate to inflamed lymph nodes and produce large amounts of type I interferon. Nat. Med. 5: 919.

11. Piali, L., Weber, C., LaRosa, G., Mackay, C. R., Springer, T. A., Clark-Lewis, I. and Moser, B., 1998, The chemokine receptor CXCR3 mediates rapid and shearresistant adhesion-induction of effector T lymphocytes by the chemokines IP10 and Mig. Eur. J. Immunol. 28: 961.

12. Svensson, H., Cederblad., B., Lindahl, M. and Alm., G., 1996, Stimulation of natural interferon-alpha/beta-producing cells by Staphylococcus aureus. J. Interferon Cytokines Res 16: 7.

13. Siegel, F. P., Kadowaki, N., Shodell, M., Fitzgerald-Bocarsly, P. A., Shah., K, Ho., S., Antonenko, S. and Liu, Y. J., 1999, The nature of the principle type I interferon-producing cells in human blood. Science 284: 1835.

14. Kadowaki, N., Antonenko, S., Lau, J. Y. and Liu, Y. J., 2000, Natural interferon alpha/beta-producing cells link innate and adaptive immunity. J. Exp. Med. 192: 219.

15. Cella Marina, F. F., Antonio,Lanzavecchia, A. and Colonna, M., 2000, Plasmacytoid dendritic cells activated by influenza virus and CD40L drive a potent Th1 polarization. Nature Immunology 1: 305.

16. Fitzgerald-Bocarsly, P., 1993, Human natural interferon-alpha producing cells. Pharmacol. Ther. 60: 39.

17. Kohrgruber, N., Halanek, N., Groger, M., Winter, D., Rappersberger, K., Schmitt-Egenolf, M., Stingl, G. and Maurer, D., 1999, Survival, maturation and function of CD11c−and CD11c+peripheral blood dendritic cells are differentially regulated by cytokines. J. Immunol. 163: 3250.

18. Robinson, S. P., Patterson, S., English, N., Davies, D., Knight, S. C. and Reid, C. D., 1999, Human peripheral blood contains two distinct lineages of dendritic cells. Eur. J. Immunol. 29: 2769.

19. Sallusto, F. and Lanzavecchia, A., 1994, Efficient presentation of soluble antigen by cultured human dendritic cells is maintained by granulocyte/macrophage colony-stimulating factor plus interleukin 4 and down-regulated by tumor necrosis factor alpha. J. Exp. Med. 179: 1109.

20. Banchereau, J. and Steinman, R. M., 1998, Dendritic cells and the control of immunity. Nature 392: 245.

21. Romani, N., Gruner, S., Brang, D., Kampgen, E., Lenz, A., Trockenbacher, B., Konwalinka, G., Fritsch, P. O., Steinman, R. M. and Schuler, G., 1994, Proliferating dendritic cell progenitors in human blood. J. Exp. Med. 180: 83.

22. Grouard, G., Rissoan, M. C., Filgueira., L., Durand, I., Banchereau, J. and Liu, Y. J, 1997, The enigmatic plasmacytoid T cells develop into dendritic cells with interleukin (IL)-3 and CD40-ligand. J. Exp. Med. 185: 1101.

23. McKenzie, J. L., Egner, W., Calder, V. L. and Hart, D. N. J., 1992, Hodgkin's disease cell lines: a model for interleukin-1-independent accessory cell function. Immunol. 77: 345.

24. Kadin, M. E., 1982, Possible origin of the Reed-Sternberg cell from an interdigitating reticulum cell. Cancer Treat. Rep. 66: 601.

25. Hsu, S. M., 1990, The never ending controversies in Hodgkin's disease. Blood 75: 1742.

26. Sorg., U. R., Morse, T. M., Patton, W. N., Hock, B. D., Angus, H. B., Robinson, B. A., Colls, B. M. and Hart, D. N. J., 1997, Hodgkin's cells express CD83, a dendritic cell lineage associated marker. Pathology 29: 294-299.

27. Hock, B. D., Feamley, D. B, Boyce, A., McLellan, A. D., Sorg, R. V., Summners, K. L., Hart, D. N. J., 1999, Human dendritic cells express a 95 kDa activation/differentiation antigen defined by CMRF-56. Tissue Antigens 53: 320-334.

28. Kohler, G. and Milstein, C., 1975, Continuous cultures of fused cells secreting antibody of predefined specificity. Nature 256: 495-497.

29. Tjissen, P., 1990, Practice and theory of enzyme immunoassays. Laboratory Techniques in Biochemistry and Molecular Biology, Elsevier, Amsterdam, New York, Oxford 117-121.

30. Hodgson, J., 1991, Making monoclonals in microbes. Biotechnology 9: 423-425.

31. Berenson, R. J., Bensinger, W. I., Kalamasz, D., Positive selection of viable cell populations using avidin-biotin immunoadsorption. Journal of Immunological Methods 91: 11.

32. Van Voorhis, W. C., Hair, L., Steinman, K. and Kaplan, G., 1982, Human dendritic cells. J. Exp. Med. 155: 1172.

33. Knight, S. C., Farrant, J., Bryant, A., Edwards, A. J., Burman, S., Lever, A., Clark, J. and Webster, A. D. B., 1986. Non-adherent low density cells from human peripheral blood contain dendritic cells and monocytes both with vieled morphology. Immunology 57: 595.

34. Young, J. W. and Steinman, R. M., 1988, Accessory cell requirements for the mixed leukocyte reaction and polyclonal mitogens as studied with a new technique for enriching blood dendritic cells. Cellular Immunology 111: 167.

35. Boyam, A., 1983, Isolation of human blood monocytes with Nycodenz, a new non-ionic iodinated gradient medium. Scand. J. Immunogy 17: 429-436.

1. An immuno-interactive molecule comprising an epitope-binding region wherein said epitope is immunodetectably present in stimulated, including partially stimulated, DC and is substantially not immunodetectable in non-stimulated DC.

2. An immuno-interactive molecule according to claim 1 wherein the immuno-interactive molecule is an antibody or an epitope-binding fragment thereof.

3. An immuno-interactive molecule according to claim 2 wherein the antibody is a monoclonal antibody.

4. An immuno-interactive molecule according to claim 2 or 3 wherein the antibody is an IgG or IgM antibody.

5. An immuno-interactive molecule according to any one of claims 1 to 4 wherein the immunodetectable epitope is on plasmacytoid or myeloid DC stimulated in the presence of GM-CSF and/or IL-3 or other cytokine or functional equivalent.

6. An immuno-interactive molecule according to claim 5 wherein the immuninteractive molecule interacts with antigen CMRF-58 (Ag CMRF-58).

7. An immuno-interactive molecule according to claim 6 wherein the immuno-interactive molecule is monoclonal antibody CMRF-58 (mAb CMRF-58) produced by hybridoma CMRF-58 deposited at Deutsche Sammlung Von Mikroorganismen und Zellkuturen GmbH (DSMZ) under Accession Number DSM ACC2434.

8. An isolated mAb CMRF-58 or preparation of mAb CMRF-58 antibodies or derivatives thereof which antibody or antibodies specifically interact with an epitope on a subset of plasmacytoid DC but substantially does not interact with antigens recognized by mAb CMRF-44, anti-CD83 antibodies and/or mAb CMRF-56.

9. An immunopotentiating composition comprising a population of DC having a molecule immunodetectably present in activated DC but which molecule is substantially not immunodetectable in non-activated DC, said composition further comprising an antigen capable of generating a protective immunological response to a disease in an animal susceptible to such disease in an animal such as a mammal including a human.

10. A composition comprising an epitope-binding region of a molecule wherein said molecule is immunodetectably present in stimulated, including partially stimulated, DC and is substantially not immunodetectable in non-stimulated DC.

11. A composition according to claim 9 or 10 wherein the immuno-interactive molecule is an antibody or an epitope-binding fragment thereof.

12. A composition according to claim 11 wherein the antibody is a monoclonal antibody.

13. A composition according to claim 11 or 12 wherein the antibody is an IgG or IgM antibody.

14. A composition according to any one of claims 9 to 13 wherein the immunodetectable epitope is on plasmacytoid or myeloid DC stimulated in the presence of GM-CSF and/or IL-3 or other cytokine or functional equivalent.

15. A composition according to claim 14 wherein the immuninteractive molecule interacts with antigen CMRF-58 (Ag CMRF-58).

16. A composition according to claim 15 wherein the immuno-interactive molecule is monoclonal antibody CMRF-58 (mAb CMRF-58) produced by hybridoma CMRF-58 deposited at Deutsche Sammlung Von Mikroorganismen und Zellkuturen GmbH (DSMZ) under Accession Number DSM ACC2434.

17. An isolated mAb CMRF-58 or preparation of mAb CMRF-58 which antibody or antibodies specifically interact with an epitope on a subset of plasmacytoid DC but substantially does not interact with antigens recognized by mAb CMRF-44, anti-CD83 antibodies and/or mAb CMRF-56.

18. An immunomodulating composition comprising agents selected from:
(i) mAb CMRF-58;
(ii) Ag CMRF-58;
(iii) Ag CMRF⁺ cells;
(iv) a modulator of Ag CMRF-58-mAb CMRF-58 interaction
or derivatives or recombinant, synthetic or hybrid forms thereof
which composition further optionally comprises an antigen capable of generating a protective immune response to an infectious agent in an animal such as a mammal including a human.

19. A method for the treatment or prophylaxis of an animal to a disease condition, said method comprising administering to said animal an effective amount of an immunomodulating composition, said composition comprising agents selected from:
(i) mAb CMRF-58;
(ii) Ag CMRF-58;
(iii) Ag CMRF-58⁺ cells;
(iv) a modulator of Ag CMRF-58-mAb CMRF-58 interaction
or derivatives or recombinant, synthetic or hybrid forms thereof
which composition further optionally comprises an antigen capable of generating a protective immune response to an infectious agent in an animal such as a mammal including a human.

20. An antagonist or agonist comprising a molecule capable of inhibiting or facilitating mAb CMRF-58-Ag CMRF-58 interaction.

21. Ag CMRF-58 in isolated form or in a preparation of DC, said antigen having an ability to bind mAb CMRF-58.

22. A method of manipulating an immune response to facilitate organ transplantation to reduce the risk of rejection of a donated organ upon transplantation or to manipulate the immune response to reduce graft failure and graft versus host disease as a consequence of allogeneic bone marrow transplantation, which method comprises the step of administering Ag CMRF-58⁺ depleted DC from the organ donor to said recipient or an antagonist of Ag CMRF-58 interaction with mAb CMRF-58.

23. A method according to claim 22 wherein the antagonist of Ag CMRF-58 is mAb CMRF-58 or a antigen-binding fragment thereof or a recombinant, synthetic or hybrid form thereof.
